(19) United States
(12) Patent Application Publication
(10) Pub. No.: US 2004/0034094 A1
(43) Pub. Date: Feb. 19, 2004

(54) Vitamin C stabilized topical formulation

(76) Inventor: Shyam K. Gupta, Scottsdale, AZ (US)

Correspondence Address: Tod R. Nissle, Esq., TOD R. NISSLE, P.C., P.O. Box 55630, Phoenix, AZ, 85078, US

(21) Appl. No.: 10/223,671

(22) Filed: Aug. 16, 2002

(51) Int. Cl. A61K 31/375; A61K 7/00

(52) U.S. Cl. 514/474; 424/401

(57) ABSTRACT

A topical formulation produces a surprising result by stabilizing the ascorbic acid in the formulation even though a significant amount of water is present in the formulation. The formulation includes at least one surfactant and at least one water miscible organic solvent and enables water soluble compositions to be incorporated in the formulation.

This invention relates to compositions that include ascorbic acid.

More particularly, the invention relates to ascorbic acid compositions that stabilize ascorbic acid.

In a further respect, the invention relates to ascorbic acid compositions that stabilize ascorbic acid without requiring that the composition have a low pH.

In another respect, the invention relates to a stabilized ascorbic acid composition that does not rely on a non-aqueous organic to stabilize the ascorbic acid by protecting it from oxygen, light, or water.

In still a further respect, the invention relates to a stabilized ascorbic acid composition that utilizes water to stabilize the composition.

The following terms used herein have the meanings set forth below.

Absorb. To swallow up, suck up by capillary, osmotic, solvent or chemical action.

Adsorb. To take up by physical or chemical forces of molecules of gas, of dissolved substances, or of liquids or by the surfaces of solids or liquids with which they are in contact.

Colloid. A substance that (a) apparently is dissolved, but diffuses slowly through a membrane; or (b) is in a fine state of subdivision so particles are too small to be visible in an ordinary optical microscope and do not settle, or settle slowly.

Dispersion. An emulsion or suspension. Comprises the dispersed substance and the medium it is dispersed in.

Emulsion. Intimate mixture of two incompletely miscible liquids.

Hydrophilic. Strong affinity for water.

Hydrophobic. Weak affinity for water.

Lipophilic. Strong affinity for fats or other lipids.

Miscible. Capable of mixing in any ratio without separation of the two phases. The mixture formed by a miscible liquid or solid is a solution.

Miscible oil. Hydrocarbon oil with emulsifiers. Forms a milky emulsion in water.

Oleophilic. Strong affinity for oils.

Organic. Being, containing, or relating to carbon compounds, especially in which hydrogen is attached to carbon whether derived from living organisms or not.

Organic solvent. A solvent including a carbon compound. Examples include, without limitation, glycerin, PEG-6 (Polyethylene glycol 300), and Mpdiol glycol.

Silicone. An organic siloxane, especially any of a large group of polymerized organic siloxanes that are unusually stable over a wide temperature range, that are obtained as oily fluids, resins and elastomers convertible into greases and other compounds, coatings, and rubbers, and that are used chiefly in waterproofing, lubrication, and electric insulation.

Silicone derivative. A silicone with one or more attributes beneficial to skin. Such attributes include emolliency, moisturization, skin smoothing, wrinkle reduction, skin softening, etc. Examples of silicone derivations, include, without limitation, most organosilicones, organic siloxanes, and their cross polymer (e.g., dimethicone, dimethicone copolyol, cetyl dimethicone copolymer, cetyl dimethicone, stearyl dimethicone, stearoxydimethicone, behenoxydimethicone, alkyl methicone, amodimethicone, dimethicone alkyl betaine, cyclomethicone, polydimethylsiloxane, diphenyldimethyl polysiloxane, silicone elastomers, cyclomethicone and dimethicone crosspolymer, Jeesilc 6056, Dow Corning 2501).

Solution. A solid, liquid, or gas mixed homogeneously with a liquid.

Solvent. A substance capable of or used in dissolving or dispersing one or more other substances, especially a liquid component of a solution present in greater amount than the solute.

Surfactant. In general, a surfactant is a substance useful for its cleansing, wetting, dispersing, or similar powers. Each of such functions are performed by a surfactant due to its ability to reduce surface tension. More specifically, as used herein, a surfactant is an ingredient that reduces the surface tension of water. Examples of surfactants include, without limitation, Tauranol 1-78-6, Tauranol ws conc., Lathanol LAL, Dow Corning 2501, Hydramol PGDS.

Suspension. Particles mixed in a fluid or a solid, but undissolved.

Water miscible organic solvent. An organic solvent that can be mixed with water in any ratio without separation of the water from the organic solvent. In the practice of the invention, the preferred (but not required) water miscible organic solvents are those commonly used in cosmetic applications, for example, glycerin, ethylene glycol, propylene glycol, butylene glycol, hexylene glycol, pyrrolidone, N-methylpyrrolidone, dimethyl sulfoxide, dimethyl sulfone, polyethylene glycol, polypropylene glycol, methylpropanediol, and similar solvents.

Various approaches are known for stabilizing ascorbic acid in various cosmetic, dermatological, and other compositions.

One such approach for slowing the degradation of ascorbic acid advocates placing ascorbic acid in low pH solutions. For example, one such composition incorporates ascorbic acid in an aqueous-alcoholic solution consisting of at least 80% water and having a pH less than 3.5. Another similar composition comprises an acidic aqueous ascorbic acid solution including an oxyethylenated sorbitan ester. This solution has a pH of 3.4. Still another similar composition comprises a water in oil emulsion containing ascorbic acid. This emulsion has a pH of 3.5 or less. A disadvantage of these compositions is that a pH of 3.5 or less is well below the normal pH of the epithelium of an individual. Applying such a composition can irritate the skin.

Another way of stabilizing ascorbic acid is to use a coating technique.

A further stabilization technique is to granulate the ascorbic acid. This technique, along with the coating technique mentioned above, tends to be expensive and may cause degradation of the ascorbic acid. Further, ascorbic acid granules can be impractical when used in compositions that are applied to the skin.

Still another stabilization technique utilizes metal salts of phosphorylated ascorbic acid in a cosmetic composition. The pH of the composition is 8 to 9. This pH is significantly higher than the normal pH of skin.

Still a further stabilized composition comprises small crystals of vitamin C in a carrier that is essentially water free and includes less than 0.5% water. Eliminating water from a composition is one commonly used approach for increasing the stability of ascorbic acid in the composition.

Yet another stabilized composition comprises ascorbic acid in the form of a fat-soluble fatty acid ester in a carrier.

Yet a further stabilized composition comprises ascorbic acid in a water in oil emulsion.

Yet still a further stabilized composition consists of particles dispersed in an oil or other non-aqueous phase. The particles include a water-soluble or water-dispersible polymer, ascorbic acid, water, and a surfactant. While oil tends shield ascorbic acid from oxygen and water, the oil, when applied to the skin, produces a feel that typically is not preferred by customers.

The foregoing approaches for stabilizing ascorbic acid are set forth in U.S. Pat. No. 6,103,267 to Mitchnick et al.

A further approach for stabilizing ascorbic acid is to seal an aqueous solution of ascorbic acid tightly in a container to prevent air from reaching the solution. See U.S. Pat. No. 6,238,713.

Another approach for stabilizing ascorbic acid is to incorporate only small amounts, typically less than 1%, of ascorbic acid into a composition. See, for example, U.S. Pat. No. 6,136,330.

It would be highly desirable to provide a vitamin C stabilized formulation that would not have an unusually low pH or an unusually high pH, that would not require granules of ascorbic acid, that would not require expensive coating techniques, that would not require the formulation be sealed in an air tight container, that would not require that only small amounts of ascorbic acid be utilized, and that would not require the use of an oil or other hydrophobic composition.

I have discovered an improved vitamin C stabilized formulation and method for making the same. The formulation avoids the use of oils, minimizes the importance of the pH of the formulation, allows the incorporation of an aqueous ascorbic acid solution in the formulation, does not require packaging the formulation in air tight containers, allows the use of large amounts of vitamin C, and does not require the use of granules or expensive coatings.

The vitamin C stabilized topical formulation of the current invention comprises ascorbic acid, water, a surfactant, and a miscible organic solvent. The formulation is particularly suited for, but not limited to, use in cosmetic and medical fields as a composition to be applied externally to the skin and hair of an individual. A silicone can be added to the formulation.

The amount of ascorbic acid in the formulation is 5% to 30% by weight, preferably 10% to 15% by weight. A particular advantage of the vitamin C stabilized formulation of the invention is that relatively large amounts of ascorbic acid up to about 30% by weight can be incorporated in the formulation. If the amount of ascorbic acid is in excess of 30%, the amount of water required becomes excessive. Consequently, quantities of ascorbic acid greater than about 30% are presently not practical in the practice of the invention. It is theorized that this is the case because as more vitamin C is utilized, more water is required. And, the amount of water appears limited by the amount of water-miscible organic solvent required.

The amount of surfactant in the formulation is 1% to 30%, preferably 20% to 30% by weight. It is possible that the amount of surfactant in the formulation can be up to 40% by weight, but concentrations of surfactant greater than 30% increase the risk that the surfactant may gel out. Less than 20% by weight of surfactant is acceptable, but the foaming properties of the formulation are not as good. The surfactant is preferably hydrophilic, and absorbs a portion of the water and vitamin C in the formulation. The water absorption characteristic of the surfactant is important in the practice of the invention because when water is bound with a hydrophilic or water-absorbing ingredient, the pH of the formulation ordinarily is not that important with respect to the stability of the vitamin C.

The amount of the water miscible organic solvent in the formulation is 1% to 80%, preferably 10% to 50% by weight. The use of a water miscible organic solvent is believed critical in the practice of the invention because it facilitates the intermixing of water—which contains vitamin C—with the organic solvent. Further, it is preferred that the organic solvent be water absorbent and absorb at least a portion of the water and vitamin C in the formulation. The water absorption characteristic of the organic solvent is, as is the case with the surfactant, important in the formulation of the invention because when water is bound with a hydrophilic or water-absorbing ingredient, the pH of the formulation ordinarily is not that important with respect to the stability of the vitamin C. The water absorption characteristic is also important because it facilitates the water miscible characteristic of the organic solvent.

The amount of water in the formulation is from 1% to 25%, preferably 5% to 15%. As a general rule, excluding water from ascorbic acid increases the stability of the ascorbic acid. Water facilitates the oxidation and instability of vitamin C. However, in the invention water is desirable and required. Surprisingly, water helps stabilize the formulation. It is theorized, although not proven, that the water solubilizes the vitamin C and thereby facilitates the mixing of the vitamin C in the water miscible organic solvent, or, in the silicone derivative.

The proportion of water to water-miscible organic solvent in the formulation appears to be important in terms of vitamin C stability and is in the range of 1:2 to 1:6, preferably 1:3 to 1:5. This proportion is believed important (although this belief has not presently been proven) because there must be sufficient water-miscible organic solvent to absorb and/or mix with water containing vitamin C to stabilize the vitamin C.

When used, the amount of silicone in the formulation is from 0.1% to 30%, preferably 1% to 10% by weight. The use of a silicone is not required, but is important because a silicone appears to enhance the stability of the vitamin C in the formulation.

Since the formulation of the invention can include water, one particular advantage of the invention is that ingredients containing water in their own composition can be utilized in preparing the formulation. Such ingredients might well not be suitable for prior art processes that require anhydrous compositions or manufacturing conditions. One particular advantage of using such ingredients in the practice of the invention is that the ingredients may supply a part of or all of the water required in the formulation, obviating the need of adding water in a separate step during the manufacture of the formulation.

The inclusion of water in formulations of the invention also permits the ready use of other water soluble or water dispersible ingredients including, by way of example and not limitation, other vitamins, anti-inflammatory agents, anesthetics, analgesics, enzymes, UV-absorbers, antiperspirants, deodorants, colorants, hydroxy acids, skin lightening agents, emollients, medications, antibiotics, antifungal agents, and insect repellents. If desired, such ingredients need not be water soluble or water dispersible.

Examples of surfactants that can be utilized are anionic, amphoteric, nonionic and cationic surfactants. Examples of anionic surfactants include, without limitation, soaps, alkyl sulfates, anionic acyl sarcosinates, methyl acyl taurates, N-acyl glutamates, acyl isethionates, alkyl phosphate esters, ethoxylated alkyl phosphate esters, alkyl sulfosuccinates, trideceth sulfates, protein condensates, mixtures of ethoxylated alkyl sulfates, and the like.

The following examples are presented by way of illustration, and not limitation, of the invention. In each example, the amount or proportion of each ingredient is in weight percent unless otherwise indicated.

### EXAMPLE I

The following ingredients are provided.

| Ingredient | Weight Percent |
|---|---|
| Glycerin (water miscible organic solvent) | 48.300 |
| Methyl paraben | 0.200 |
| Vitamin C | 25.000 |
| Phenoxyethanol | 0.900 |
| Tauranol I-78-6 (Sodium Cocoyl | 20.000 |
| Isethionate) (surfactant) | |
| Tauranol ws conc. (Sodium Methyl | 5.000 |
| Cocoyl Taurate) (surfactant) | |
| Actiplex 2789 (Extract of various plants) | 0.100 |
| Fragrance | 0.500 |

All of the ingredients are mixed to form a composition. The mixture is heated and stirred at 60 to 70 degrees C. until the mixture is homogenous, i.e., for about five to ten minutes. The homogeneous mixture is cooled to room temperature. The colorants are admixed in the homogenous mixture to form a vitamin C stabilized formulation. It is not necessary to add water during preparation of the vitamin C stabilized formulation because the surfactants contain sufficient water. The vitamin C stabilized formulation is used as a facial, hair, and body cleanser.

### EXAMPLE II

The following ingredients are provided.

| Ingredient | Weight Percent |
|---|---|
| PEG-6 (water miscible organic solvent) | 50.900 |
| Methyl paraben | 0.200 |
| Vitamin C | 20.000 |
| Phenoxyethanol | 0.900 |
| Tauranol I-78-6 (Sodium Cocoyl | 20.000 |
| Isethionate) (surfactant) | |
| Lathanol LAL (Sodium Lauryl | 5.000 |
| Sulfoacetate) (surfactant) | |
| Fragrance | 1.000 |
| Jeesilc 6056 (A proprietary blend | 2.000 |
| of Polydimethylsiloxane, Dimethyl | |
| Siloxane, Polyethylene Oxide | |
| Lauryl Ether, and water) | |
| FDC Yellow #6 | qs |
| FDC Yellow #5 | qs |

All of the ingredients, with the exception of the colorants (FDC Yellow #5, #6) are mixed to form a composition. The mixture is heated and stirred at 60 to 70 degrees C. until the mixture is homogenous, i.e., for about five to ten minutes. The homogeneous mixture is cooled to room temperature. The colorants are admixed in the homogenous mixture to form a vitamin C stabilized formulation. It is not necessary to add water during preparation of the vitamin C stabilized formulation because the surfactants contain sufficient water. The vitamin C stabilized formulation is applied to the skin. The silicone derivative provides emolliency.

Examples of anionic non-soap surfactants are, without limitation, the alkali metal salts of organic sulfate having in their molecular structure an alkyl radical containing from about 8 to about 22 carbon atoms and a sulfonic acid or sulfuric acid ester radical.

Examples of Zwitterionic surfactants are, without limitation, derivatives of aliphatic quaternary ammonium, phosphonium, and sulfonium compounds, in which the aliphatic radicals can be straight chain or branched and wherein one of the aliphatic substitutents contains from about 8 to 18 carbon atoms and one contains an anionic water-solubilizing group, e.g, carboxy, sulfonate, sulfate, phosphate, or phosphonate.

Examples of amphoteric surfactants are, without limitation, derivatives of aliphatic secondary and tertiary amines in which the aliphatic radical can be straight chain or branched and wherein one of the aliphatic substituents contains from about 8 to about 18 carbon atoms and one contains an anionic water solubilizing group, e.g., carboxy, sulfonate, sulfate, phosphate, or phosphonate.

Examples of cationic surfactants are, without limitation, stearyidimenthylbenzyl ammonium chloride; dodecyltrimethyl ammonium chloride;nonylbenzylethyldimethyl ammonium nitrate; and tetradecylpyridinium bromide.

Nonionic surfactants include, without limitation, compounds produced by the condensation of alkylene oxide groups (hydrophilic in nature) with an organic hydrophobic compound, which may be aliphatic or alkyl aromatic in nature, for example, the polyethylene oxide condensates of alkyl phenols.

### EXAMPLE III

The following ingredients are provided.

| Ingredient | Weight Percent |
|---|---|
| Deionized water | 20.000 |
| Vitamin C | 15.000 |
| Mpdiol glycol (Methylpropanediol) | 51.000 |
| (water miscible organic solvent) | |
| Dow Corning 2501 (surfactant/silicone | 10.000 |
| derivative) (Bis-PEG-18 Methyl | |
| Ether Dimethyl Silane) | |
| Structure Plus (Acrylates/Aminoacrylates/ | 4.000 |
| C10-30 Alkyl PEG-20 Itaconate Copolymer) | |

The vitamin C is dissolved in the deionized water to produce an aqueous solution of vitamin C. The remaining three ingredients are mixed together in a separate tank to produce a supplemental mixture. The aqueous solution is added to the tank and admixed with the supplemental mixture to produce a homogeneous vitamin C stabilized formulation. The formulation comprises a clear gel. The gel is applied to the epidermis on the face and body.

### EXAMPLE IV

The following ingredients are provided.

| Ingredient | Weight Percent |
|---|---|
| Deionized water | 15.000 |
| Vitamin C | 11.000 |
| Mpdiol glycol (Methylpropanediol) | 59.000 |
| (water miscible organic solvent) | |
| Dow Corning 2501 (Bis-PEG-18 Methyl | 4.000 |
| Ether Dimethyl Silane) | |
| (surfactant/silicone derivative) | |
| Phenoxyethanol | 0.700 |
| Liquapar (Parabens) | 0.300 |
| Hydromol PGDS (PEG-90 Diisostearate) | 10.000 |

The vitamin C is dissolved in the deionized water to produce an aqueous solution of vitamin C. The remaining three ingredients are mixed together in a separate tank to produce a supplemental mixture. The supplemental solution is heated to 50 to 60 degrees C. to produce a clear solution. The aqueous solution is added to the tank and admixed with the supplemental mixture to produce a homogeneous vitamin C stabilized formulation. The formulation is cooled to room temperature. A clear syrupy liquid is obtained. The formulation is applied to the epidermis on the face, including under the eyes.

### EXAMPLE V

The liquid of Example IV is stored at room temperature in a container. A lid is laid across the top of the container to slow evaporation. The lid does not prevent ambient air from slowly entering the container. After six months the liquid is still clear and colorless.

### EXAMPLE VI

The liquid of Example IV is stored at room temperature in a container. A lid is laid across the top of the container to slow evaporation. The lid does not prevent ambient air from slowly entering the container. After one year the liquid is still clear and colorless.

### EXAMPLE VII

The liquid of Example IV is stored at room temperature in a container. A lid is laid across the top of the container to slow evaporation. The lid does not prevent ambient air from slowly entering the container. After eighteen months the liquid is still clear and colorless.

### EXAMPLE VIII

The liquid of Example IV is stored at room temperature in a container. A lid is laid across the top of the container to slow evaporation. The lid does not prevent ambient air from slowly entering the container. After twenty-four months the liquid is a very light yellowish brown.

### EXAMPLE IX

Example V to VIII is repeated except that the white paste of Example III is used in place of the liquid of Example IV. Similar results are obtained. The color of the paste remains white at the six and twelve month marks, but is off white to light yellowish brown at the eighteen and twenty-four month marks.

### EXAMPLE X

A sample formulation is prepared according to the Example set forth in U.S. Pat. No. 6,103,267 to Mitchnick et al. The formulation is stored at room temperature in a container. A lid is laid across the top of the container to slow evaporation. The lid does not prevent ambient air from slowly entering the container. After one year the liquid is dark brown.

Having described the presently preferred embodiments and best mode of the invention in such terms as to enable those of skill in the art to understand and practice the invention, I claim:

1. A vitamin C stabilized topical formulation comprising
(a) 5% to 30% by weight ascorbic acid;
(b) 1% to 25% by weight water;
(c) at least one surfactant; and,
(d) at least one water miscible organic solvent.

2. A vitamin C stabilized topical formulation comprising
(a) 5% to 30% by weight ascorbic acid;
(b) 1% to 25% by weight water;
(c) 0.1% to 30% by weight of at least one cosmetic silicone derivative;
(d) 1% to 30% by weight of at least one surfactant; and,
(e) 1% to 80% by weight of at least one water miscible organic solvent.

3. A vitamin C stabilized topical formulation comprising
(a) 5% to 30% by weight ascorbic acid;
(b) 1% to 30% by weight of at least one surfactant; and,
(c) 1% to 80% by weight of at least one water miscible organic solvent;
at least one of a pair comprising said surfactant and said solvent including water, said water comprising 1% to 25% by weight of said formulation.

4. A vitamin C stabilized topical formulation comprising
(a) 5% to 30% by weight ascorbic acid;
(b) at least one surfactant;
(c) at least one miscible organic solvent; and,
(d) at least one ingredient other than said surfactant and said solvent including water, said water comprising 0.1% to 25% by weight of said formulation.

5. A vitamin C stabilized topical formulation including
(a) 5% to 30% by weight ascorbic acid;
(b) 1% to 25% by weight water;
(c) at least one water-absorbing surfactant; and,
(d) at least one water-absorbing miscible organic solvent.

6. A vitamin C stabilized topical formulation comprising
(a) 5% to 30% by weight ascorbic acid;
(b) 1% to 25% by weight water; and,
(c) at least one surfactant absorbing a portion of said water and said ascorbic acid; and,
(d) at least one water miscible organic solvent absorbing a portion of said water and said ascorbic acid.

7. A method of forming a vitamin C stabilized formulation comprising the steps of
(a) mixing water and ascorbic acid to form an aqueous vitamin C solution;
(b) mixing at least one water-absorbing surfactant and at least one water-absorbing water miscible organic solvent to form a water-absorbing mixture; and,
(c) admixing said aqueous vitamin C solution and said water-absorbing mixture such tat at least a portion of said aqueous vitamin C solution is absorbed by said water-absorbing mixture.

8. A method of forming a vitamin C stabilized formulation comprising the steps of
(a) mixing at least one water-absorbing surfactant and at least one water-absorbing water miscible organic solvent to form an aqueous composition, at least one of said surfactant and said solvent including water; and,
(b) admixing ascorbic acid to said aqueous composition to produce a stabilized vitamin C composition, said stabilized vitamin C composition including from 1% to 25% by weight water provided by at least one of said surfactant and said solvent.
